(19) United States
(12) Patent Application Publication
(10) Pub. No.: US 2022/0378372 A1
(43) Pub. Date: Dec. 1, 2022

(54) WEARABLE PATCHES FOR SPORTS

(71) Applicant: LIFE PATCH INTERNATIONAL, Irvine, CA (US)

(72) Inventor: Donald BOLLELLA, Irvine, CA (US)

(21) Appl. No.: 17/885,245

(22) Filed: Aug. 10, 2022

Related U.S. Application Data
(63) Continuation of application No. PCT/US2021/017685, filed on Feb. 11, 2021.
(60) Provisional application No. 62/972,654, filed on Feb. 11, 2020.

(51) Int. Cl. A61B 5/00; A61B 5/01; A61B 5/145; A61B 5/021; A61B 5/024

(52) U.S. Cl. CPC A61B 5/6833; A61B 5/01; A61B 5/14517; A61B 5/021; A61B 5/024; A61B 5/14542

(57) ABSTRACT

A wearable patch for sports can include a patch substrate configured to support a plurality of components, and to allow the patch to be attached to a skin of a user engaged in a sporting activity. The patch can further include a sensor implemented at least partially within the patch substrate and configured to sense a condition of the user. The patch can further include a transmitter circuit in communication with the sensor and configured to transmit information representative of the sensed condition to a location external to the wearable patch.

### CROSS-REFERENCE TO RELATED APPLICATION(S)

This application is a continuation of International Application No. PCT/US2021/017685 filed Feb. 11, 2021, entitled WEARABLE PATCHES FOR SPORTS, which claims priority to U.S. Provisional Application No. 62/972,654 filed Feb. 11, 2020, entitled WEARABLE PATCHES FOR SPORTS, the benefits of the filing dates of which are hereby claimed and the disclosures of which are hereby expressly incorporated by reference herein in their entirety.

### BACKGROUND

### Field

The present disclosure relates to wearable patches for sports.

### Description of the Related Art

Various sporting activities are enjoyed and pursued by many for exercise, enjoyment, competition, etc. Regardless of the reason, it is desirable to improve performance for a number of reasons, such as reduction of injuries, improved personal satisfaction, improved team performance, etc.

### SUMMARY

In accordance with a number of implementations, the present disclosure relates to a wearable patch that includes a patch substrate configured to support a plurality of components, and to allow the patch to be attached to a skin of a user engaged in a sporting activity. The patch further includes a sensor implemented at least partially within the patch substrate and configured to sense a condition of the user. The patch further includes a transmitter circuit in communication with the sensor and configured to transmit information representative of the sensed condition to a location external to the wearable patch.

In some embodiments, the condition can include, for example, a temperature of the skin, a perspiration level of the skin, a heart rate, a blood condition (such as oxygen level of a sample of blood obtained from the user, an oxygen level of the user obtained in a non-invasive manner or a lactic acid level of a sample of blood obtained from the user), a body movement or a body position, an ambient temperature or an ambient lighting.

In some embodiments, the patch substrate can include an adhesive layer configured to allow the wearable patch to stick to the skin of the user. In some embodiments, the adhesive layer can be configured to be substantially water resistant.

In some embodiments, the transmit circuit can include an antenna. In some embodiments, the transmitter circuit can include a radio-frequency identification (RFID) circuit.

In some implementations, the present disclosure relates to a method for monitoring an athlete engaged in a sporting activity. The method includes attaching a patch to a skin of the athlete, sensing a condition of the athlete with a sensor implemented in the patch while the athlete is actively engaged in the sporting activity, and transmitting information representative of the sensed condition to a location external to the patch.

In some teachings, the present disclosure relates to a system for monitoring an athlete. The system includes a patch configured to be attached to a skin of the athlete and sense a condition of the athlete. The patch is further configured to transmit information representative of the sensed condition. The system further includes a monitor external to the patch. The monitor is configured to receive the information from the patch through a communication link and generate an output.

In some embodiments, the communication link can be a direct link implemented with a wireless signal. In some embodiments, the communication link can include an intermediate component such that a first link provides communication between the patch and the intermediate component and a second link provides communication between the intermediate component and the monitor. The first link between the patch at the intermediate component can be implemented with a wireless signal. The patch can include a radio-frequency identification (RFID) circuit configured to receive an interrogation signal and transmit the information in response to receipt of the interrogation signal. The intermediate component can include an RFID reader configured to transmit the interrogation signal to the patch and to receive the information from the patch. The second link can include a wireless router implemented to provide communication between the RFID reader and the monitor.

In some embodiments, the monitor can be configured to provide the output to a person other than the athlete. The person other than the athlete can be, for example, a coach.

In some embodiments, the intermediate device can be configured to be positioned on or near the athlete. The intermediate device can be configured to, for example, be worn on a wrist or waist of the athlete, or be a part of an equipment (such as a helmet) being used by the athlete.

In some embodiments, wherein the monitor can be configured to provide the output to the athlete. The monitor can be configured to be positioned on or near the athlete. The monitor can be configured to be worn on a wrist or waist of the athlete.

In some embodiments, the system can further include a sensor configured to a part of a sporting equipment used by the athlete to measure a performance related parameter associated with the use of the sporting equipment. The sensor can be configured to provide information corresponding to the measured performance related parameter to the monitor. The sensor can include, for example, an RFID circuit and/or an accelerometer.

In some implementations, the present disclosure relates to a kit for monitoring an athlete. The kit includes a plurality of patches implemented in a packaged format, with each patch including a patch substrate configured to support a plurality of components, and to allow the patch to be attached to a skin of the athlete. Each patch further includes a sensor implemented at least partially within the patch substrate and configured to sense a condition of the athlete, and a transmitter circuit in communication with the sensor and configured to transmit information representative of the sensed condition. The kit further includes a printed instruction configured to facilitate use of the patch on the athlete.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 depicts a patch device that can be worn by an athlete during a sporting activity.

FIG. 2 shows an example form factor of a patch having one or more features as described herein.

FIG. 3 shows another example form factor of a patch having one or more features as described herein.

FIG. 4 shows that in some embodiments, the patch of FIGS. 2 and 3 can include one or more sensing functionalities to support monitoring of an athlete during a sporting activity.

FIG. 5 shows an example of a system that can be implemented utilizing a patch having one or more features as described herein.

FIG. 6A shows that in some embodiments, a wireless communication link can be implemented in the system of FIG. 5.

FIG. 6B shows that in some embodiments, the system of FIG. 5 can include a first communication link between a patch and one or more intermediate devices, and a second communication link between the intermediate device(s) and a monitor.

FIG. 7 shows a system that can be a more specific example of the system of FIG. 6B.

FIG. 8 shows that in some embodiments, a patch having one or more features as described herein can be implemented in a bracelet format.

FIG. 9 shows that in some embodiments, a patch having one or more features as described herein can be implemented in a patch format.

FIG. 10 shows an example of a patch having a plurality of layers configured to provide a fluidic analysis functionality.

FIG. 11 shows another example of a patch having a plurality of layers configured to provide a fluidic analysis functionality.

FIG. 12 shows a sports-related device implemented to be worn with a band.

FIG. 13 shows a sports-related device implemented to be worn on a wrist.

FIG. 14 shows a sports-related device that is an equipment specific to a sport being engaged by a user wearing a patch.

FIGS. 15A to 15E show non-limiting examples of sporting equipment with each having one or more sensors.

FIG. 16 shows a sporting activity where an athlete is engaged in a lacrosse game or practice.

FIG. 17A depicts an example of a system configured to accommodate an application setting such as in team sports.

FIG. 17B depicts another example of a system configured to monitor a plurality of sporting activities utilizing a plurality of patches.

FIG. 18 shows that in some embodiments, one or more patches having one or more features as described herein can be provided in a packaged format for easier use.

FIG. 19 shows an example of a packaged format having a support sheet with a plurality of patches secured thereto.

FIG. 20 shows an enlarged side sectional view of an example support sheet that can be utilized to hold a plurality of patches.

### DETAILED DESCRIPTION OF SOME EMBODIMENTS

The headings provided herein, if any, are for convenience only and do not necessarily affect the scope or meaning of the claimed invention.

Disclosed are examples related to systems, methods and devices for monitoring an athlete during sporting activities. For the purpose of description, it will be understood that an athlete may be a professional athlete, an amateur athlete, a recreational athlete, or simply anyone engaged in physical activity.

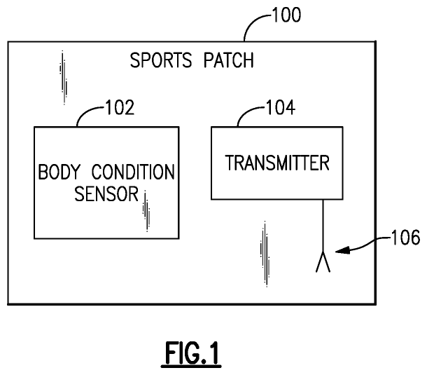

FIG. 1 depicts a patch device 100 that can be worn by an athlete during a sporting activity. In some embodiments, the patch 100 can include a body condition sensor 102 and a transmitter 104 configured to transmit information about one or more body conditions sensed by the sensor 102. Such transmission of information can be supported by an antenna 106 in communication with the transmitter 104.

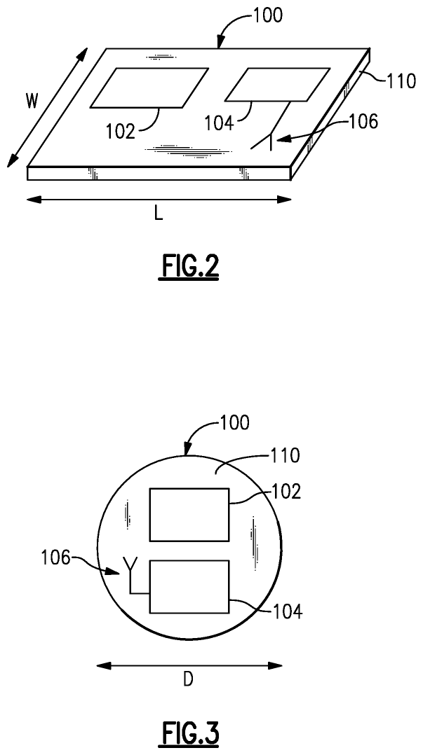

FIGS. 2 and 3 show non-limiting examples of how a patch 100 can be implemented in different form factors. For example, FIG. 2 shows that in some embodiments, a patch 100 can have a generally rectangular shape with a length L and a width W. Such dimensions can be selected to, for example, allow application of the patch 100 on an area of the user with sufficient space (e.g., on an arm).

In another example, FIG. 3 shows that in some embodiments, a patch 100 can have an elliptical shape, such as a circular shape, with a diameter D. Such a dimension can be selected to, for example, allow application of the patch 100 on a smaller area of the user that is more discreet and/or more sensitive to body surface condition.

It will be understood that a patch 100 having one or more features as described herein can be implemented with other shapes. It will also be understood that functionalities associated with a patch having one or more features as described herein can also be implemented in other forms of wearable devices.

In the examples of FIGS. 2 and 3, each patch 100 can include a patch substrate 110 configured to provide wearable functionality and to support a number of components. Examples related to such wearable functionality and support functionality can be found in U.S. Pat. No. 9,133,024 titled PERSONAL DIAGNOSTIC DEVICES INCLUDING RELATED METHODS AND SYSTEMS, which is expressly incorporated by reference in its entirely, and its disclosure is to be considered part of the specification of the present application.

In the examples of FIGS. 2 and 3, each patch 100 is shown to include a body condition sensor 102, a transmitter 104, and an antenna 106, similar to the example of FIG. 1. In some embodiments, at least the transmitter portion of the patch 100 can be include an RFID (radio-frequency identification) circuitry configured to support transfer of information between the patch 100 and a device external to the patch 100.

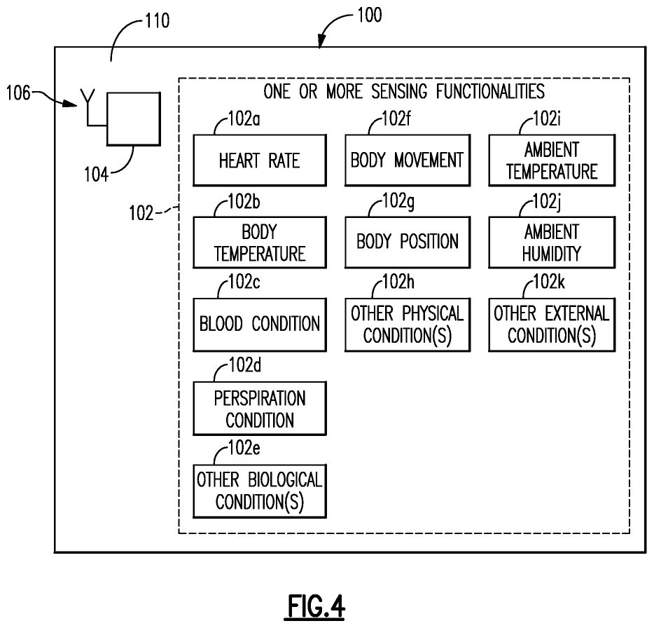

FIG. 4 shows that in some embodiments, the patch 100 of FIGS. 2 and 3 can include one or more sensing functionalities to support monitoring of an athlete during a sporting activity. For example, a body condition sensor can be configured to sense one or more biological conditions, one or more physical conditions, and/or one or more external conditions.

The one or more biological conditions can include, for example, heart rate, body temperature, blood condition (e.g., blood oxygen level, lactic acid level, etc.), perspiration condition, etc. Accordingly, the heart rate can be sensed by a heart rate sensor 102a; the body temperature can be sensed by a body temperature sensor 102b; the blood condition can be sensed by a blood condition sensor 102d; the perspiration condition can be sensed by a perspiration condition sensor 102e; and the other body condition(s) can be sensed by one or more respective sensors 102e.

The one or more physical conditions can include, for example, body movement, body position, and/or other physical condition(s). Accordingly, the body movement can be sensed by a body movement sensor 102f; the body position can be sensed by a body position sensor 102g; and the other physical condition(s) can be sensed by one or more respective sensors 102h.

The one or more external conditions can include, for example, ambient temperature, ambient lighting condition, and/or other external condition(s). Accordingly, the ambient temperature can be sensed by an ambient temperature sensor 102i; the ambient humidity condition can be sensed by an ambient humidity sensor 102j; and the other external condition(s) can be sensed by one or more respective sensors 102k.

In some embodiments, a patch having one or more features as described herein can be worn by an athlete during a sporting activity. Such a patch can be configured to be secured to a portion of the athlete (e.g., on a skin surface) so as to allow sensing of one or more desired body conditions while not interfering with the sporting activity. Since sports include a wide variety of physical activities in many different settings and conditions, in some embodiments, a patch can be configured for use in a specific sport. In some embodiments, a patch can also be configured for use in a number of different sports, since many sports can have common features such as motion, perspiration, increased heart rate, muscle usage, etc.

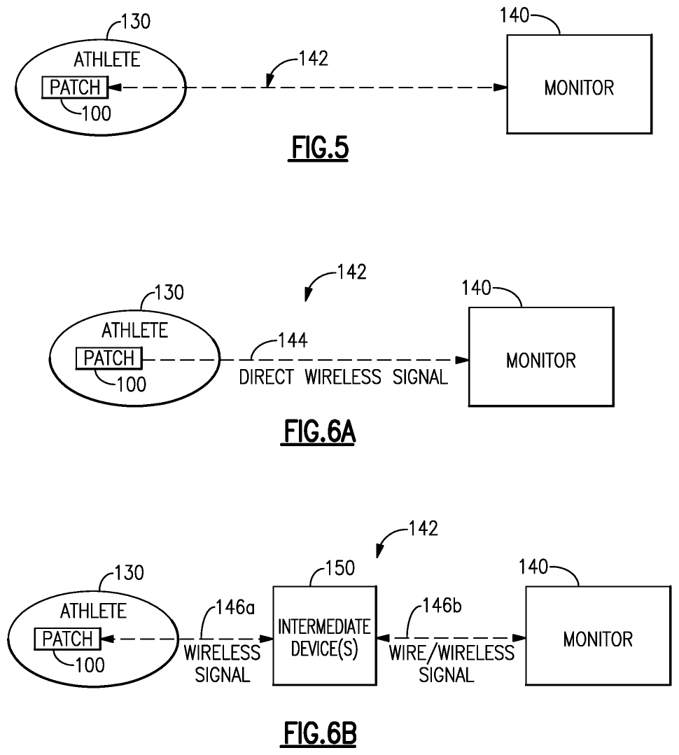

As described herein, a patch having one or more features as described herein can include a communication component to support transmission of information such as sensor data, and/or to support reception of information such as control signals. FIG. 5 shows an example of a system that can be implemented to utilize such a communication functionality. For example, a patch 100 having one or more features as described herein is shown to be worn by a user 130 such as an athlete. Such a patch can be in a communication link 142 with a monitor 140. In some embodiments, such a monitor can be configured to provide some or all of monitoring functionality, alert functionality, and control functionality.

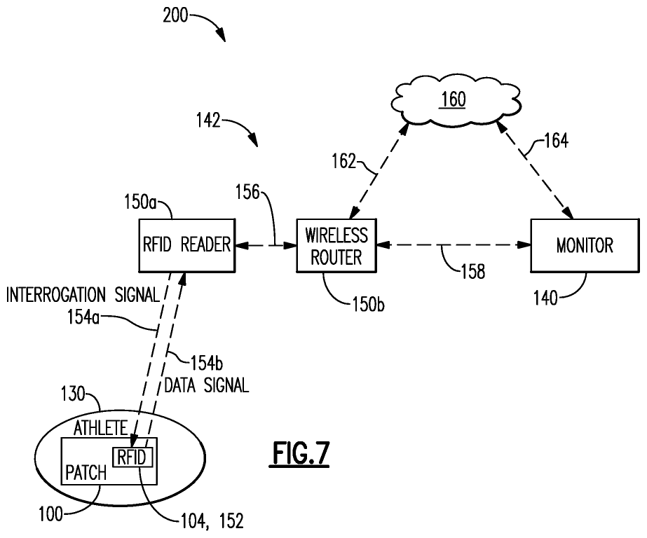

As an example, FIG. 6A shows that in some embodiments, the communication link 142 of FIG. 5 can include a wireless signal 144 transmitted by the patch 100 to the monitor 140. In some embodiments, such a wireless signal can be provided directly from the patch 100 to the monitor 140, and can include one or more sensed information such as the examples of FIG. 4.

In the example of FIG. 6A, it will be understood that the communication link 142 may or may not include a wireless signal (e.g., a control signal) that is transmitted by the monitor 140 and received by the patch 100. A patch having an RFID functionality is an example where such a wireless control signal may be utilized.

In another example, FIG. 6B shows that in some embodiments, the communication link 142 of FIG. 5 can include a first communication link 146a between a patch 100 and one or more intermediate devices 150, and a second communication link 146b between the intermediate device(s) 150 and a monitor 140. It is noted that a wire on or near an active athlete is not preferred; accordingly, in some embodiments, the first communication link 146a can be a wireless link. In such a configuration, the wireless link 146a between the patch 100 and an intermediate device 150 can include a wireless signal provided from the patch 100 to the intermediate device 150, and such a wireless signal can include one or more sensed information such as the examples of FIG. 4.

In some embodiments, the wireless signal received by the intermediate device 150 can be sent to the monitor 140 without further processing, be processed by the intermediate device 150 and resulting information sent to the monitor, or some combination thereof. It is noted that either or both of wired and wireless link may be utilized for the second communication link between the intermediate device 150 and the monitor 140.

In the example of FIG. 6B, it will be understood that the first communication link 146a may or may not include a wireless signal (e.g., a control signal) that is transmitted by, for example, the intermediate device 150 and received by the patch 100. A patch having an RFID functionality is an example where such a wireless control signal may be utilized.

In the example of FIG. 6B, it will also be understood that the second communication link 146b may or may not include a signal (e.g., a control signal) that is transmitted the monitor 140 and received by the intermediate device 150. Such a control signal may be for controlling of the intermediate device 150, the patch 100, or some combination thereof.

FIG. 7 shows a system 200 that can be a more specific example of the system of FIG. 6B. In the example of FIG. 7, a patch 100 having one or more features as described herein can include an RFID circuit 152 as a part of a communication component 104. As described herein, such a patch can be worn by an athlete 130 during a sporting activity.

In the example of FIG. 7, a monitor 140 ban be configured to provide a patch-monitoring functionality by, for example, a mobile application software (also referred to as an app) running in the monitor 140, as well as communication components associated with the monitor 140. In some embodiments, such a monitor can be, for example, a portable wireless device such as a tablet, a portable computer, a smartphone, etc.

In the example of FIG. 7, the one or more intermediate devices 150 of FIG. 6B can include an RFID reader 150a and a wireless router 150b. In some embodiments, the RFID reader 150a can be configured to be deployed within range to an athlete being monitored so as to allow RFID communication with the patch). In some embodiments, such an RFID reader can be configured to communicate directly with the monitor (e.g., wirelessly) or, as shown in FIG. 7, through the wireless router 150b.

In the context where the wireless router 150b is being utilized, communication between the wireless router 150b (and thus the RFID reader 150a and the patch 100) and the monitor 140 can be provided with a direct link 158 or through internet 160. For the direct link 158 example, such a link can be through, for example, a wireless local area network (WLAN). In some embodiments, such a direct link can be suitable for an arrangement where the monitor 140 and the wireless router 150b are within an operating range.

For the link through the internet 160, such a link can be achieved through a communication link 162 between the wireless router 150b and an internet service provider, and a communication link 164 between a communication service provider and the monitor 140. It is noted that the foregoing internet service provider and the communication service provider may or may not be the same entity.

For example, the internet service provider can be an entity that communicates with the wireless router 150b, and the communication service provider can be an entity that provides a cellular communication service. In such an example where cellular communication service is utilized by the monitor 140, one can see that the patch-monitoring functionality can be achieved even when the monitor 140 is at a remote location.

In another example, the internet service provider can be an entity that communicates with the wireless router 150b, and the communication service provider can include a different WLAN connection for the monitor 140. Accordingly, one can see that the patch-monitoring functionality can be achieved wherever the monitor is able to connect to a WLAN.

In the example of FIG. 7, communication between the monitor 140 and the patch 100 can include communication links between the RFID reader 150a and the patch 100. For example, the RFID reader 150a can provide an interrogation signal 154a to the RFID circuit 152 of the patch 100. In response to such an interrogation signal, the RFID circuit 152 can transmit a data signal 154b to the RFID reader 150a. Such a data signal can include one or more sensed information obtained previously to receipt of the interrogation signal 154a, obtained approximately contemporaneously with the receipt of the interrogation signal 154a, obtained after the receipt of the interrogation signal 154a, or some combination thereof.

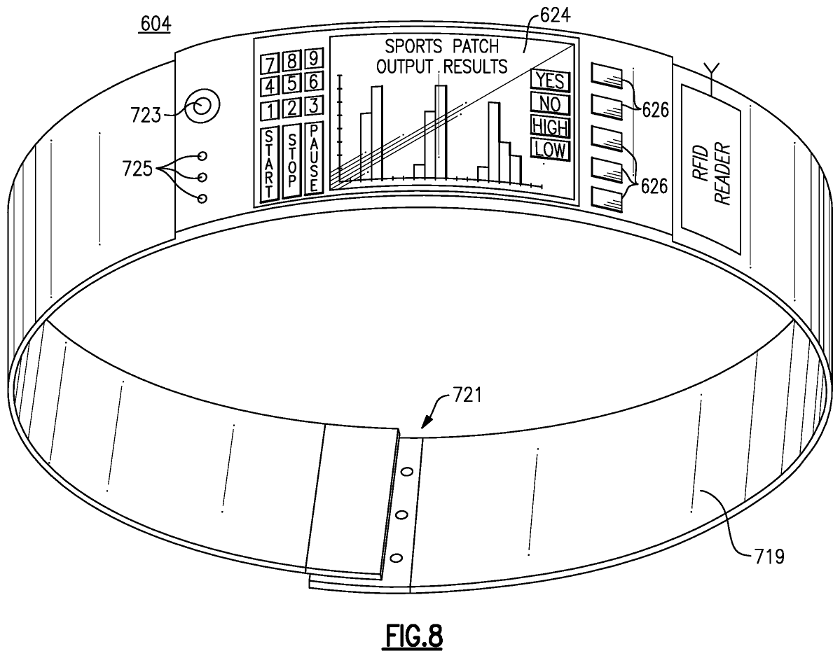

FIG. 8 shows that in some embodiments, a patch having one or more features as described herein can be implemented in a bracelet format 604, and can include a radio frequency transmitter 723 as described herein. The device 604 as shown can also include connection indicator lights 725 which may be enabled to blink or otherwise emit light to indicate a proper wireless connection between the device 604 and an external device.

In the example of FIG. 8, the device 604 can include an RFID circuit configured to provide either or both of receive and transmit functionalities, including some or all of the examples described herein.

The bracelet 604 of FIG. 8 is shown to be provided with a band 719 and a clasp 721 so that a user may wear the band around the wrist or ankle, for example. The band 719 may be made of a flexible, stretch material to provide good holding force between the device and the user, or may be made of a hard material such as stainless steel or aluminum.

FIG. 8 shows that in some embodiments, the device 604 can also include a video display monitor 624 and one or more individual fixed-display results windows 626, implemented to provide information for the user. It is also noted that in the device 604 of FIG. 8, the RF transmitters 723 may include both transmission capabilities as well as receiving capabilities to thereby establish two-way communication between the device 604 and an external device, such as in the examples described herein.

Additional examples related to the device 604 of FIG. 8 can be found in the above-referenced U.S. Pat. No. 9,133,024.

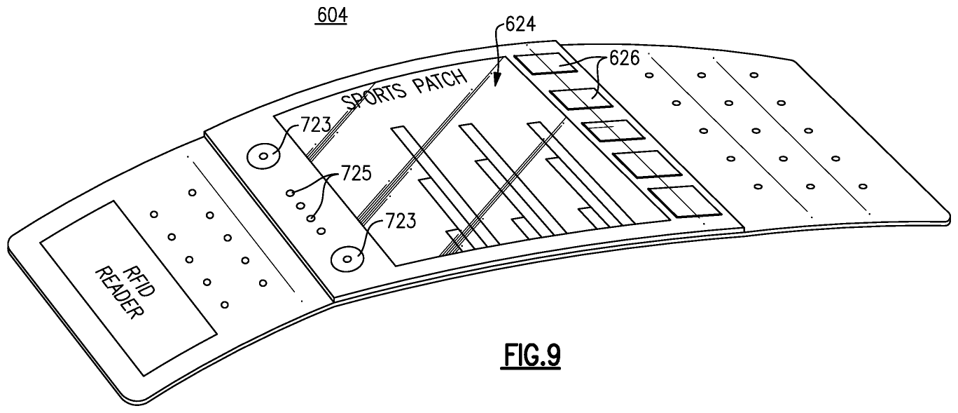

FIG. 9 shows that in some embodiments, a patch having one or more features as described herein can be implemented in a patch format 604, and can include a radio frequency transmitter 723 as described herein. The device 604 as shown can also include connection indicator lights 725 which may be enabled to blink or otherwise emit light to indicate a proper wireless connection between the device 604 and an external device.

In the example of FIG. 9, the device 604 can include an RFID circuit configured to provide either or both of receive and transmit functionalities, including some or all of the examples described herein.

FIG. 9 shows that in some embodiments, the device 604 can also include a video display monitor 624 and one or more individual fixed-display results windows 626, implemented to provide information for the user. It is also noted that in the device 604 of FIG. 9, the RF transmitters 723 may include both transmission capabilities as well as receiving capabilities to thereby establish two-way communication between the device 604 and an external device, such as in the examples described herein.

Additional examples related to the device 604 of FIG. 9 can be found in the above-referenced U.S. Pat. No. 9,133,024.

FIG. 10 shows an example of a patch 604 having a plurality of layers configured to provide a fluidic analysis functionality. More particularly, a perspective cut-away pictorial view of the patch 604 shows internal assemblies of micro-fluidic circuits, assay results detectors, and electronic signal processing components according to the teaching hereof. As described herein, such an analysis functionality can be configured to analyze a sampled fluid such as blood.

In the example of FIG. 10, the patch 604 can include a sample acquisition layer, a fluid processing layer, a results detection layer, and a wash buffer reservoir layer. Some or all of such layers can be configured to support a fluidic circuit that may include a sample metering chamber 804, a capture chamber 806, an analysis chamber 812, a waste chamber 814, and a wash buffer reservoir 816. Such chambers or reservoirs may be operatively connected to each other and may include one or more vents to prevent air blockages with the fluidic circuit. The connections and flow between chambers/reservoirs may be controlled by, for example, melt plugs, detent pressure valves, melt valves, and pinch valves in any desired combination to allow controlled movement of the samples and buffers between the different chambers/reservoirs. In an example configuration, the capture zone 812 may be provided with several waste cambers 814 disposed around the periphery thereof to aid in the collection of waste fluids.

In the example of FIG. 10, the device 604 can be configured to allow sampling of fluid such as blood in a minimally invasive manner. For example, minimally invasive tubules, lancets, or micro-probes can be provided, and be operatively connected to reservoir openings 664, the blood metering chamber 804, the capture chamber 806, a micro-wire emitter 820, the analysis chamber 812, the waste chamber 814, a detector 822 (e.g., a micro-wire detector), and the wash buffer reservoir 816. Operations involving some or all of such parts of the device 604 can be supported by, for example, a signal processor 642, the controller 690, an output video display monitor 624, and individual fixed-display results windows 626.

In the example of FIG. 10, the device 604 can include an RFID circuit configured to provide either or both of receive and transmit functionalities, including some or all of the examples described herein.

Additional examples related to the device 604 of FIG. 10 can be found in the above-referenced U.S. Pat. No. 9,133,024.

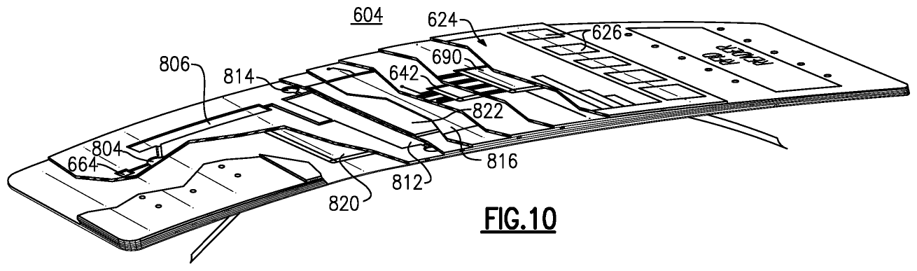

FIG. 11 shows another example of a patch 604 having a plurality of layers configured to provide a fluidic analysis functionality. As described herein, such an analysis functionality can be configured to analyze a sampled fluid such as blood for.

In the example of FIG. 11, the patch 604 can be configured to allow analyte quantification (e.g., quantification of small molecules). The patch 604 may include a sample acquisition layer 616, a sample processing layer 880, and an electrochemical detector layer or alternatively, (or in combination therewith), a photo detector-layer. Sample processing can be carried out in a biocompatible lateral flow matrix preloaded with analysis reagents as described in further detail in the above-referenced U.S. Pat. No. 9,133,024.

In the example of FIG. 11, the sample processing layer 880 can include a particular micro-fluidic circuit with individual fluid components. The sample processing layer 880 may include one micro-fluidic circuit or may alternatively include a plurality of micro-fluidic circuits formed therein. Alternatively, the device 604 may include a plurality of sample processing layers 880 each with one or more micro-fluidic circuits. In this manner, a plurality of blood samples may be taken over time by using flow control. Each of these separate blood samples may then be processed in real time (or approximately real time) in a separate micro-fluidic circuit.

For example, the device 604 may include 12 micro-fluidic circuits either all formed and situated on one sample processing layer 880, or each formed and situated on separate individual sample processing layers 880 for a total for 12 such sample processing layers. As understood, the 12 circuits would be preferably fluidly isolated from each other. Alternatively, the 12 micro-fluidic circuits may be formed and situated in pairs, groups of three, or groups of four, for example, with each pair or group then being formed on one such layer 880. The various layers are then assembled accordingly to function as intended. In this specific embodiment, each of the 12 micro-fluidic circuits would be fluidly connected to a respective lancet 659 as described herein. With blood sampling and fluid control directed and timed by the controller 642, a melt plug associated with each lancet 659 may be activated at a specific time to put a respective metering chamber 804 in fluid communication with its respective lancet 659 to thereby draw a fresh sample of blood by capillary action at a specific predetermined time. With 12 circuits and 12 corresponding lancets 659, for example, a user may wear the device 604 for a twenty-four hour period (or a duration of sporting activity) with a blood sample taken every two hours (or every predetermined or programmed time interval).

In some embodiments, the foregoing micro-fluidic circuit can include a blood metering chamber 804, a separate blood separator zone and a capture zone. In some embodiments, the separate blood separator zone and the capture zone may be combined into a single zone or chamber with serial processing of the sample fluid through a biocompatible lateral flow matrix or membrane which may be comprised of separate pads or segments, or alternatively comprised of a single lateral flow matrix provided with various gradations for different fluid processing results along the length thereof.

In some embodiments, the device 604 may include a pre-loaded supply reservoir of material that can be released into the user. Such release of material can be controlled by, for example, a software stored in a logic controller 690 or signal processor 642 configured to direct a control system to release a determined dose of the material. To release the material, a supply duct may be provided with a one-way valve of the flexible detent lip type. Also, a plug can be used to hold the material in the supply reservoir. Such a plug is preferably not dissolvable upon contact with liquid because it needs to hold the material in the supply reservoir until directed by the control system to release the dosage in the reservoir. Such a control system can utilize an electric signal generated by the control system to send a current to the plug which is pre-wired with a resistive element and designed to melt when current is supplied to the resistive element embedded in the plastic type plug material. In this manner, the material from the supply can be released in a controlled, timed fashion. Metering the release flow and closing the channel or supply duct may also be achieved. The dosage is preferably controlled by the size of the supply reservoir wherein a simple discharge of the entire volume from the reservoir is initiated when needed or desired. The reservoirs can be of different volumes and the desired volume selected by the control system by sending the melt current to the reservoir with the correct volume.

In the example of FIG. 11, the device 604 is shown to include the above-described layers configured to provide one or more assay functionalities. As described herein, minimally invasive tubules, lancets, or micro-probes 659, reservoir openings 664, a blood metering chamber 804, a material reservoir 895, supply lancets 897, a signal processor 642, a controller 690, an output video display monitor 124, and individual fixed-display results windows 126 may be provided and configured to provide respective functionalities.

In the example of FIG. 11, the device 604 can include an RFID circuit configured to provide either or both of receive and transmit functionalities, including some or all of the examples described herein.

Additional examples related to the device 604 of FIG. 11 can be found in the above-referenced U.S. Pat. No. 9,133,024.

In the example of FIG. 10, the patch device 604 can be configured to perform an assay of sampled blood to measure, for example, lactic acid level in the sampled blood. In the example of FIG. 11, the patch device 604 can be configured to perform an assay of sampled blood to measure, for example, oxygen level in the sampled blood. It will be understood that a patch device having one or more features as described herein can be configured to measure other blood-related properties.

As described herein, a patch can be configured to communicate with a monitor (e.g., 140 in FIGS. 5-7) directly (e.g., as in FIG. 6A) or through an intermediate device (e.g., as in FIGS. 6B and 7). FIGS. 12-14 show non-limiting examples of devices that can function as a monitor and/or an intermediate device.

For example, FIG. 12 shows a sports-related device 900 implemented to be worn with a band (e.g., with a waist band such as a belt, or an arm band). Such a sports-related device 900 can be a portable electronic device configured to function as an intermediate device 150 and/or a monitor device 140, with functionalities as described herein. In some embodiments, the sports-related device 900 can include an RFID reader configured to communicate with a patch having an RFID functionality.

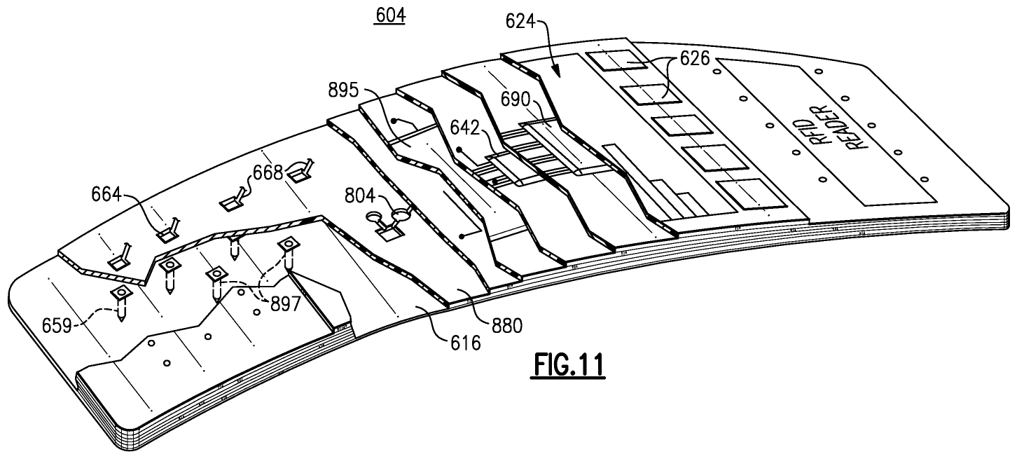

For example, FIG. 13 shows a sports-related device 900 implemented to be worn on a wrist. Such a sports-related device 900 can be a portable electronic device configured to function as an intermediate device 150 and/or a monitor device 140, with functionalities as described herein. In some embodiments, the sports-related device 900 can include an RFID reader configured to communicate with a patch having an RFID functionality.

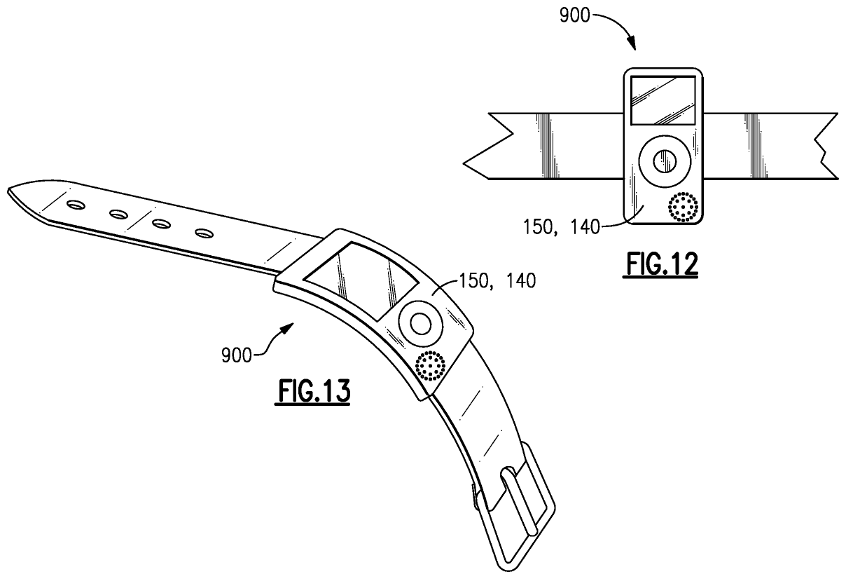

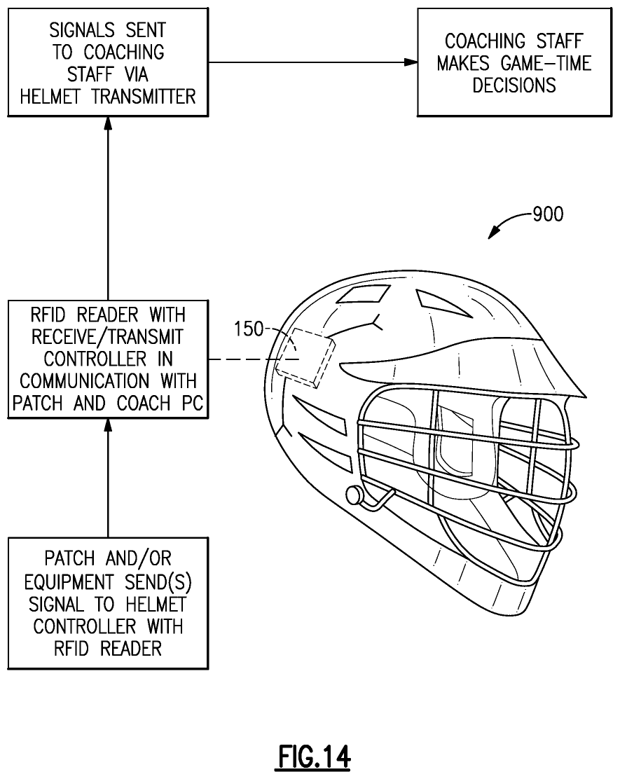

In yet another example, FIG. 14 shows a sports-related device 900 that is an equipment specific to a sport being engaged by a user wearing a patch. In the example of FIG. 14, the sports-related device 900 is a lacrosse helmet that includes an intermediate device 150. In some embodiments, such an intermediate device can be attached to an existing helmet in a secure and safe manner, be an integral part of a helmet, or any combination thereof. In some embodiments, such an intermediate device can support a communication link between the patch worn by the user and a monitoring device away from the user. In some embodiments, the sports-related device 900 can include an RFID reader configured to communicate with a patch having an RFID functionality.

FIG. 14 also shows an example of the foregoing communication link between a patch worn by a user (e.g., a player) and a monitoring device away from the patch. As shown, a patch and/or a related or similar equipment can send a signal to a controller of the intermediate device 150 attached to the helmet 900 utilizing an RFID circuit. The intermediate device 150 can include a communication circuit (e.g., an RFID circuit) configured to provide receive/transmit functionalities. Thus, the intermediate device 150 can be in communication with the patch as well as with a computing device associated with a monitoring person (e.g., a coach). Accordingly, signals can be sent from the patch to the computing device being used by the coach, via the intermediate device 150 attached to the helmet 900. Based on such signals, the computing device can provide information about the player; and such information can be utilized for post-game analysis or game-time decisions.

In some implementations, one or more sensors can be provided on a sporting equipment, and such sensor(s) can be utilized with a patch having one or more features as described herein and worn by a user. Information obtained from such equipment sensor(s) can be combined with information obtained from the patch to, for example, enhance monitoring functionality for the user.

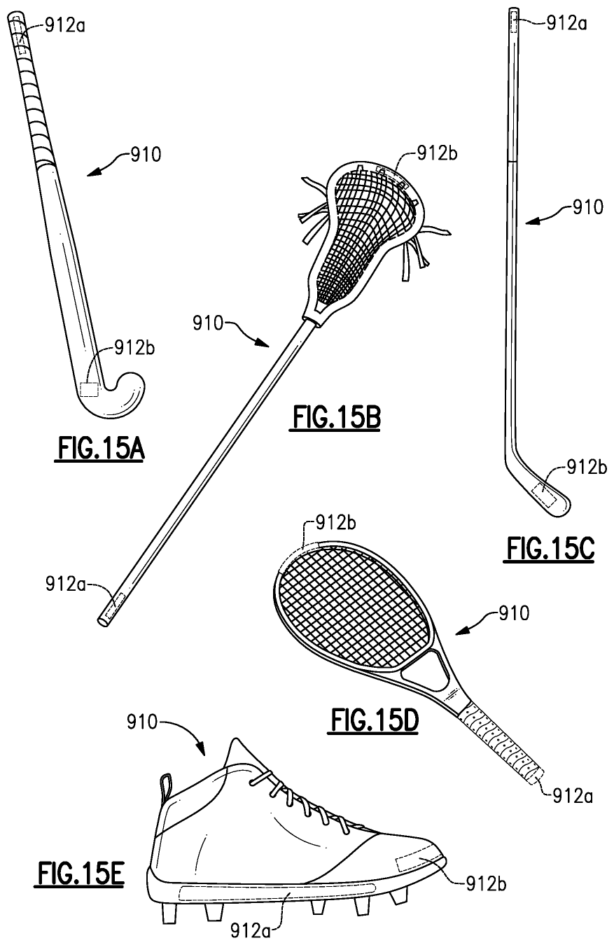

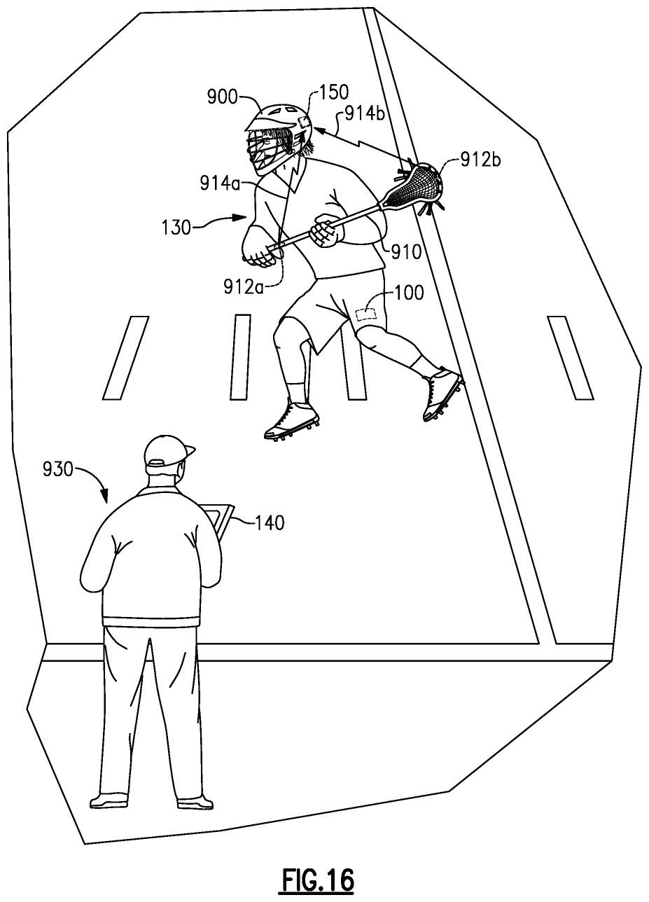

FIGS. 15A-15E show non-limiting examples of sporting equipment with each having one or more sensors. It will be understood that a sensor for such a sporting equipment can be attached to an existing equipment in a secure and safe manner, be an integral part of an equipment, or any combination thereof.

FIG. 15A shows a field hockey stick 910 configured to be used in field hockey sport. In the example shown, the field hockey stick 910 includes a first sensor 912a implemented at or near one end (e.g., grip end), and a second sensor 912b implemented at or near the other end (e.g., head end).

FIG. 15B shows a lacrosse stick 910 configured to be used in lacrosse sport. In the example shown, the lacrosse stick 910 includes a first sensor 912a implemented at or near one end (e.g., grip end), and a second sensor 912b implemented at or near the other end (e.g., head end).

FIG. 15C shows a hockey stick 910 configured to be used in hockey sport. In the example shown, the hockey stick 910 includes a first sensor 912a implemented at or near one end (e.g., grip end), and a second sensor 912b implemented at or near the other end (e.g., blade end).

FIG. 15D shows a tennis racket 910 configured to be used in tennis sport. In the example shown, the tennis racket 910 includes a first sensor 912a implemented at or near one end (e.g., handle end), and a second sensor 912b implemented at or near the other end (e.g., frame end).

FIG. 15E shows a cleat 910 configured to be used in sports such as soccer, lacrosse, field hockey, football, baseball, etc. In the example shown, the cleat 910 includes a first sensor 912a implemented along a side of the cleat, and a second sensor 912b implemented at an end (e.g., front end) of the cleat.

In the various examples of FIGS. 15A-15E, each example equipment is shown to include two sensors. However, it will be understood that more or less number of sensors can be utilized.

In some embodiments, a sensor associated with a sporting equipment (e.g., the examples of FIGS. 15A-15E) can be configured to measure one or more motion related parameters. For example, an accelerometer can be utilized to measure motion of the corresponding sensor, and thus the respective portion where the sensor is positioned. Accordingly, one or more motion related information can be obtained while the sporting equipment is being used by the user.

In some embodiments, a sensor associated with a sporting equipment (e.g., the examples of FIGS. 15A-15E) can include an RFID circuit configured to provide a communication functionality similar to an RFID functionality in some of the patch examples described herein. In some embodiments, such an RFID circuit (of the sensor) can allow the sensor to be associated appropriately with a patch being used by the user. Accordingly, information from the sporting equipment can be associated properly with information from the patch worn by the user.

In some implementations, combining of the information from the sporting equipment with the information from the patch worn by the user can provide a number of desirable results. Such results can include, for example, analysis of performance (e.g., kinematics of the sporting equipment) as a function of condition of the user.

In the examples described herein in reference to FIGS. 15A-15E, the sensors are described in the context of being associated with respective patches worn by users. It will be understood that in some embodiments, a sensor on a sporting equipment can be utilized by itself and a monitor without necessarily having a patch being worn by a user.

It is noted that a system having a patch as described herein can be utilized in many different sporting activities to monitor one or more conditions of an athlete and related performance levels. As one can appreciate, such monitoring functionality can allow appropriate actions or adjustments to be made with the athlete to thereby improve the overall performance and safety of the athlete.

For example, FIG. 16 shows a sporting activity where an athlete 130 is engaged in a lacrosse game or practice. The athlete 130 is shown to be wearing a patch 100 having one or more features as described herein. Such a patch can measure one or more conditions associated with the athlete (e.g., one or more of the conditions of FIG. 4), and provide information related to such condition(s) to a monitor 140 being monitored by a coach or a coaching staff member 930.

In the example of FIG. 16, the patch 100 can communicate with the monitor 140 through an intermediate device 150 associated with a helmet 900. Such communication between the patch 100 and the monitor 140 can be achieved as described herein (e.g., as in FIGS. 6B and 7).

In the example of FIG. 16, a lacrosse stick 910 being used by the athlete 130 is depicted as having first and second sensors 912a, 912b. Sensed information (e.g., accelerometer information) from such sensors are shown to be transmitted to the intermediate device 150 (e.g., via RFID functionality), and the intermediate device 150 can provide such information to the monitor 140 for analysis along with the athlete's condition information obtained through the patch 100.

In the example of FIG. 16, the coach 930 can take action based on the foregoing analysis of the information obtained by the monitor. For example, if the analysis shows that the athlete is becoming physically exhausted based on information from the patch 100 and/or the sensors 912a, 912b on the lacrosse stick 910, the coach 930 can have the athlete 130 replaced with another player.

In the example of FIG. 16, the sporting activity is a team sport. However, it will be understood that a similar system can be implemented in an individual sport as well. For example, suppose that a cyclist is training while wearing a patch as described herein. A training coach can be in a vehicle following the cyclist from at a distance, while monitoring the performance of the cyclist with a system as described herein.

In the foregoing example, an intermediate device can be implemented in a number of places, including, for example, on a bicycle itself, as part of a helmet, etc. If one or more equipment sensors are to be utilized, such sensor(s) can be implemented as part of, for example, cycling shoes.

Still referring to the example cycling sport, suppose that monitoring of the athlete is desired during a racing event in which the athlete is part of a team. Each team member can be equipped similarly, and a coach or a designated person can monitor the performance of each member to better adjust the team configuration during the race. For example, a pack leader can be monitored for exhaustion, and when such exhaustion is detected, the coach can instruct a change in pack leader if communication is allowed. Alternatively, the current pack leader can be the designated monitoring person, and once notified of the exhaustion condition, signal another team member to become the pack leader.

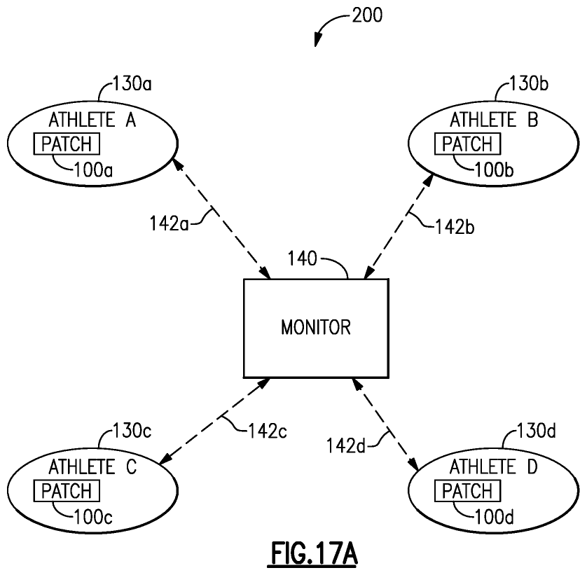

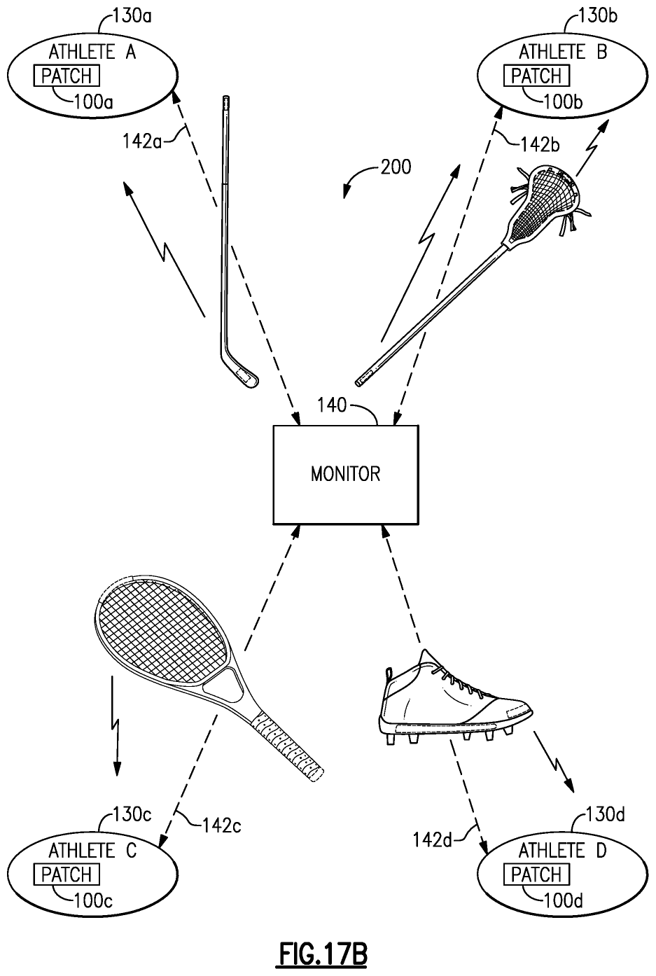

FIG. 17A depicts a system 200 configured to accommodate an application setting such as in the foregoing team sport examples. In the example of FIG. 17A, a given patch can include a unique identifier (e.g., an RFID) that allows the patch to be identified among patches that are otherwise similarly configured. Thus, the patch 100a can include a unique identifier; the patch 100b can include a unique identifier; the patch 100c can include a unique identifier; and the patch 100d can include a unique identifier.

In some embodiments, the system 200 of FIG. 17A can be configured such that a communication link between a given patch and the monitor 140 includes a unique identifier information associated with that patch. Thus, a communication link 142a can include a unique identifier information associated with the patch 100a; a communication link 142b can include a unique identifier information associated with the patch 100b; a communication link 142c can include a unique identifier information associated with the patch 100c; and a communication link 142d can include a unique identifier information associated with the patch 100d.

It is noted that in some embodiments, the system 200 of FIG. 17A can include an application where a plurality of patches are utilized for a plurality of players engaged in a common sport (e.g., a team sport such as lacrosse). The system 200 of FIG. 17A can also include an application where a plurality of patches are utilized for one or more players engaged in different sports. Such different sports can include a team sport, an individual sport, or some combination thereof.

For example, FIG. 17B shows a system where a patch 100a is shown to be worn by Athlete A (130a) playing hockey. Such a patch, with or without an equipment sensor, is shown have a communication link 142a with a monitor 140. A patch 100b is shown to be worn by Athlete B (130b) playing lacrosse; and such a patch, with or without an equipment sensor, is shown have a communication link 142b with the monitor 140. A patch 100c is shown to be worn by Athlete C (130c) playing tennis; and such a patch, with or without an equipment sensor, is shown have a communication link 142c with the monitor 140. A patch 100d is shown to be worn by Athlete D (130d) playing soccer; and such a patch, with or without an equipment sensor, is shown have a communication link 142d with the monitor 140.

In the example of FIG. 17B, the four athletes (A, B, C, D) may be a single person engaged in four different sports at different times. In such an application, the system can be useful to provide game-time or post-game analyses of the person to, for example, promote performance improvements, identify strong or weak sport skills of the person, etc.

In the example of FIG. 17B, the four athletes (A, B, C, D) may be one or more persons engage in four different sports. In such an application, the system can be useful to provide game-time or post-game analysis for each person to wearing the respective patch to, for example, promote performance improvement in the respective sport.

FIG. 18 shows that in some embodiments, one or more patches having one or more features as described herein can be provided in a packaged format 282 for easier use. Such a packaged format of patch(es) can be included in, for example, a packaged product 280. In some embodiments, the packaged product 280 can also include an instruction 284 such as a printed instruction. Such an instruction can provide information on, for example, proper and/or recommended application and use of the included patch(es).

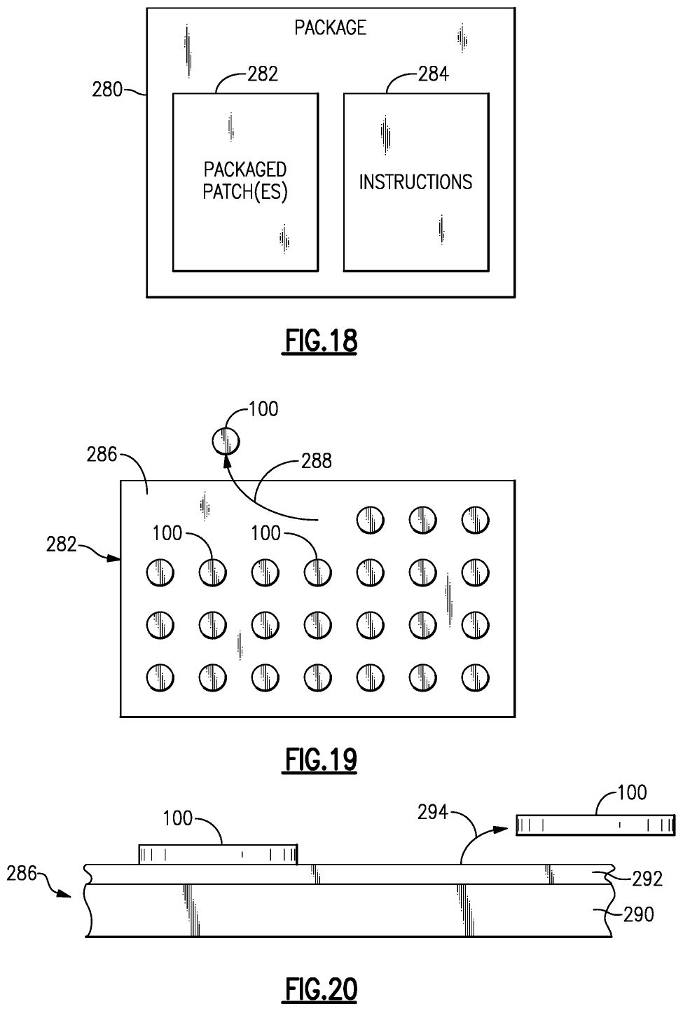

FIG. 19 shows an example of a packaged format 282 having a support sheet 286 with a plurality of patches 100 secured thereto. Such number of patches can allow one to remove (arrow 288) a patch 100 from the support sheet 286 for use. For example, one patch can be utilized during each sleeping period.

FIG. 20 shows an enlarged side sectional view of an example support sheet 286 that can be utilized to hold (until removal) a plurality of patches, similar to the example of FIG. 19. In some embodiments, the support sheet 286 can include a base layer 290 (e.g., paper, plastic, etc.) and a release layer 292. The release layer 292 can be secured to the base layer 290, and be configured to securely hold the patches 100 thereon during transport and storage phases. Assuming that a patch includes an adhesive layer for application onto the skin of a user, the release layer can further be configured to allow the patch to be removed (e.g., peeled off) cleanly for application onto the user. In the example of FIG. 20, such removal of the patch 100 from the release layer 292 is depicted as an arrow 294.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. A wearable patch comprising:
a patch substrate configured to support a plurality of components, and to allow the patch to be attached to a skin of a user engaged in a sporting activity;
a sensor implemented at least partially within the patch substrate and configured to sense a condition of the user; and
a transmitter circuit in communication with the sensor and configured to transmit information representative of the sensed condition to a location external to the wearable patch.

2. The wearable patch of claim 1 wherein the condition includes a temperature of the skin.

3. The wearable patch of claim 1 wherein the condition includes a perspiration level of the skin.

4. The wearable patch of claim 1 wherein the condition includes a heart rate.

5. The wearable patch of claim 1 wherein the condition includes a blood condition.

6. The wearable patch of claim 5 wherein the blood condition includes an oxygen level of a sample of blood obtained from the user.

7. The wearable patch of claim 5 wherein the blood condition includes an oxygen level of the user obtained in a non-invasive manner.

8. The wearable patch of claim 5 wherein the blood condition includes a lactic acid level of a sample of blood obtained from the user.

9. The wearable patch of claim 1 wherein the condition includes a body movement or a body position.

10. The wearable patch of claim 1 wherein the condition includes an ambient temperature or an ambient lighting.

11. The wearable patch of claim 1 wherein the patch substrate includes an adhesive layer configured to allow the wearable patch to stick to the skin of the user.

12. The wearable patch of claim 11 wherein the adhesive layer is configured to be substantially water resistant.

13. The wearable patch of claim 1 wherein the transmit circuit includes an antenna.

14. The wearable patch of claim 1 wherein the transmitter circuit includes a radio-frequency identification (RFID) circuit.

15. A method for monitoring an athlete engaged in a sporting activity, the method comprising:
attaching a patch to a skin of the athlete;
sensing a condition of the athlete with a sensor implemented in the patch while the athlete is actively engaged in the sporting activity; and
transmitting information representative of the sensed condition to a location external to the patch.

16. A system for monitoring an athlete, the system comprising:
a patch configured to be attached to a skin of the athlete and sense a condition of the athlete, the patch further configured to transmit information representative of the sensed condition; and
a monitor external to the patch, the monitor configured to receive the information from the patch through a communication link and generate an output.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The system of claim 16 further comprising a sensor configured to a part of a sporting equipment used by the athlete to measure a performance related parameter associated with the use of the sporting equipment.

33. The system of claim 32 wherein the sensor is configured to provide information corresponding to the measured performance related parameter to the monitor.

34. The system of claim 32 wherein the sensor includes an RFID circuit.

35. The system of claim 32 wherein the sensor includes an accelerometer.

36. (canceled)
